(19) United States
(12) Patent Application Publication
(10) Pub. No.: US 2005/0032149 A1
(43) Pub. Date: Feb. 10, 2005

(54) Enzyme catalyzed isotope labeling

(76) Inventors: Xudong Yao, Belmont, MA (US); Catherine Fenselau, Baltimore, MD (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/786,457

(22) Filed: Feb. 26, 2004

Related U.S. Application Data
(60) Provisional application No. 60/450,034, filed on Feb. 26, 2003.

(51) Int. Cl. G01N 33/53; C12Q 1/37; C12P 21/06

(52) U.S. Cl. 435/23; 435/68.1

(57) ABSTRACT

In a method of enzyme-catalyzed isotope labeling of peptides, peptides are formed from proteins digested by proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes. Thereafter isotopic atoms are incorporated into the peptides in the catalytic presence of proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes.

### CROSS-REFERENCE TO RELATED APPLICATIONS

This application claims the benefit of U.S. Provisional Application No. 60/450,034, filed Feb. 26, 2003, the subject matter of which is incorporated herein by reference in its entirety.

### BACKGROUND OF THE INVENTION

(1) Field of the Invention

The invention relates to isotope labeling of peptides and more particularly to enzyme proteolysis of proteins followed by enzyme-catalyzed isotope labeling of peptides.

(2) Description of Related Art

It is of interest to introduce stable isotope labels into peptides for a variety of applications. Foremost among these are comparative proteomics studies, where mass spectrometry is used to quantitate the relative amounts of peptides and proteins in two protein pools, e.g., from cell lines with two different health states or histories.

Other applications include preparation of isotope-labeled internal standards, facilitation of sequencing based on fragment ions in mass spectra, preparation of tracer compounds for chemical and biochemical studies.

A number of chemical reactions have been proposed, which label the parent protein, and are combined with chemical or enzymatic digestion to produce labeled peptides {(a) Gygi, S. P.; Rist, B.; Gerber, S. A.; Turecek, F.; Gelb, M. H.; Aebersold, R. Nature Biotechnol. 1999, 17, 994. (b) Munchbach, M.; Quadroni, M.; Miotto, G.; James, P. Anal. Chem. 2000, 72, 4047. (c) Geng, M.; Ji, J.; Regnier, F. E. J. Chromatogr., A 2000, 870, 295. (c) Goshe, M. B.; Conrads, T. P.; Panisko, E. A.; Angell, N. H.; Veenstra, T. D.; Smith, R. D. Anal. Chem. 2001, 73, 2578.}.

Other strategies involve the growth of organisms or cells in isotope labeled media, which lead to incorporation of isotope labels into cellular proteins. These are then isolated and cleaved into labeled peptides {Oda, Y.; Huang, K.; Croos, F. R.; Cowburn, D.; Chait, B. T. Proc. Natl. Acad. Sci. U.S.A. 1999, 96, 6591. (b) Veenstra, T. D.; Martinović, S.; Anderson, G. A.; Pa\check{s}a-Tolić, L.; Smith, R. D. J. Am. Soc. Mass Spectrom. 2000, 11, 78-82. (c) Washburn, M. P., Ulaszek, R., Deciu, C., Schieltz, D. M.; Yates, J. R. III. Anal. Chem. 2002, 74, 1650.}.

Previously reported {see (a) Yao, X.; Freas, A.; Ramirez, J.; Demirev, P. A.; Fenselau, C. Anal. Chem. 2001, 73, 2836. (b) Reynolds, K.; Yao, X.; Fenselau, C. J. Proteome Res. 2002, 1, 27.} and others {(a) Schnolzer, M.; Jedrzejewski, P.; Lehmann, W. D. Electrophoresis 1996, 17, 945. (b) Wang, Y. K.; Ma, Z.; Quinn, D. F.; Fu, E. W. Anal. Chem. 73, 2001, 3742. (c) Mirgorodskaya, O. A.; Kozmin, Y. P.; Titov, M. I.; Korner, R.; Sonksen, C. P.; Roepstorff, P. Rapid Commun. Mass Spectrom. 2000, 14, 1226.} has been use of serine proteases (trypsin, Lys-C endoprotease and Glu-C endoprotease) to cleave proteins, and simultaneously to incorporate two isotopes of O-18 from highly enriched O-18 water. Two isotopes are incorporated into every new carboxyl terminus formed, in enzyme catalysis-based cleavage and exchange reactions. The incorporation can exceed 95% if the water in which the proteolysis is carried out is enriched >95% in O-18. A mechanistic scheme is shown in FIG. 1, in which one O-18 atom is introduced when the solvent disrupts the covalent intermediate formed between the serine protease and peptide substrates. The second atom of O-18 is introduced when the enzyme rebinds the newly formed peptides and an enzyme-catalyzed oxygen exchange reaction occurs. The high level of enrichment provides a four Dalton increment between peptide pairs representing the two protein pools, which is sufficient for quantitative determinations of isotope ratios by mass spectrometers with appropriate resolution.

An equation has been published that considers the abundances of natural isotopes and provides the ratio of labeled and unlabeled peptides {see Yao, X.; Freas, A.; Ramirez, J.; Demirev, P. A.; Fenselau, C. Anal. Chem. 2001, 73, 2836, which is hereby incorporated by reference in its entirety.}. It has also been determined the analytical figures of merit to demonstrate the robustness of this approach {Reynolds, K.; Yao, X.; Fenselau, C. J. Proteome Res. 2002, 1, 27.}.

In most reports {see (a) Yao, X.; Freas, A.; Ramirez, J.; Demirev, P. A.; Fenselau, C. Anal. Chem. 2001, 73, 2836. (b) Reynolds, K.; Yao, X.; Fenselau, C. J. Proteome Res. 2002,1, 27. (c) Wang, Y. K.; Ma, Z.; Quinn, D. F.; Fu, E. W. Anal. Chem. 73, 2001, 3742. (d) Mirgorodskaya, O. A.; Kozmin, Y. P.; Titov, M. I.; Korner, R.; Sonksen, C. P.; Roepstorff, P. Rapid Commun. Mass Spectrom. 2000, 14, 1226.}, the production of O-18 coded peptides was intended to facilitate comparative proteomic measurements of protein levels in two different protein pools. The production of labeled peptides by proteolysis reactions requires relatively large amounts of O-18 water. Conditions for the proteolysis reactions must be adjusted to keep the protein substrates in solution, and are not always compatible with optimal enzyme activity. Most critically, the protein substrates have to be dehydrated in order to provide aqueous solutions >95% enriched in O-18 water. Often it is difficult to completely resolubilize the proteins.

The known methods require chemical synthesis of isotope labeled alkylating agents and chemical reactions to attach these to peptides. Other methods introduce O-18 as a by product of proteolysis reactions, which must be optimized for proteolysis, not for isotope incorporation.

### BRIEF SUMMARY OF THE INVENTION

In this invention, isotope labels are introduced into carboxylic acid groups in peptides under enzyme catalysis. ¹⁸O atoms, which are stable to chemical exchange, and can be introduced into all peptides that end in an amino acid residue that is bound by one of a variety of proteolytic enzymes. The enzyme-catalyzed isotope incorporation is conducted in a separate step from enzymatic proteolysis of proteins, under conditions that can be optimized for rapid labeling, minimal enzymatic autolysis, using a minimum of the isotope source.

Some embodiments of this invention are directed to a method of labeling peptides, comprising the steps of: a) obtaining peptides formed from proteins digested by proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes; and thereafter b) incorporating isotopic atoms into said peptides in the catalytic presence of proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes.

In some embodiments of the method of this invention, the proteolytic enzymes or mixture of enzymes in step a) is the same as the proteolytic enzymes or mixture of proteolytic enzymes in step b). In some embodiments, the proteolytic enzymes in step a) and in step b) are trypsin. In some other embodiments, the proteolytic enzymes in step a) and in step b) are a mixture of Glu-C and trypsin.

In some embodiments of this invention, the proteolytic enzymes or mixture of enzymes in step a) are different from the proteolytic enzymes or mixture of proteolytic enzymes in step b). In some of these embodiments, the proteolytic enzymes in step a) are Lys-C and in step b) are trypsin.

The proteolytic enzymes in step a) and in step b) can be obtained from any species or from thermophilic microorganisms.

In this invention, one or two isotopic atoms can be incorporated into a peptide and, in some embodiments, the isotopic atoms are ¹⁸O atoms.

In some embodiments of this invention, the step of incorporating isotopic atoms into said peptides is conducted in H₂¹⁸O.

In some embodiments of this invention, the method can further comprise mixing labeled peptides obtained from the step of incorporating of isotopic atoms with peptides that have not been labeled to obtain a mix of labeled and unlabeled peptides. In some of these embodiments, the method can further comprise analyzing the mix of labeled and unlabeled peptides by mass spectrometry. In some of these embodiments, the a ratio of labeled peptides to unlabeled peptides can be calculated with formula (I) or (II)
ratio={I₄−(M₄/M₀))×I₀−(M₂/M₀)×[I₂−(M₂/M₀)×I₀]+[I₂−(M₂/M₀)×I₀]}/I₀ (I)
ratio={I₄−(M₄/M₀))×I₀−(M₂/M₀)×[I₂−(M₂/M₀)×I₀]+½[I₂−(M₂/M₀)×I₀]}/I₀ (II)

where I₀, I₂ and I₄ are observed peak areas for a monoisotopic peak for peptides without ¹⁸O label, a peak 2 Da higher and peak 4 Da higher, and M₀, M₂ and M₄ are the theoretical peak areas for the monoisotopic peak for a peptide with a known composition, a peak 2 Da higher and peak 4 Da higher, respectively.

In some embodiments of this invention, mass spectrometry utilized can be nanospray, electrospray, LC-MS, LC-MS-MS or matrix-assisted IR or UV laser desorption ionization, high vacuum, atmospheric or low pressure, on a quadropole, quadropole ion trap, time-of-flight, ion cyclotron resonance, magnetic sector ion analyzer or a combination thereof.

In some embodiments of this invention, the step of incorporating isotopic atoms results in peptides in the catalytic presence of proteolytic enzymes being labeled greater than 90% with one or two ¹⁸O atoms. In some of these embodiments, the step of incorporating isotopic atoms results in peptides in the catalytic presence of proteolytic enzymes being labeled greater than 95% with one or two ¹⁸O atoms.

In some embodiments of this invention, the method further comprises a step of storing the peptides overnight after step a) and before step b).

Some embodiments of this invention are directed to a method of labeling peptides, comprising the steps of: digesting proteins with proteolytic enzymes in an H₂¹⁶O environment to obtain peptide fragments of said proteins, where said proteolyic enzymes are selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes; and then labeling at least a first portion of said peptide fragments by incorporating ¹⁸O atoms into said at least first portion of said peptide fragments in an H₂¹⁸O environment and in the catalytic presence of proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes.

In some embodiments of this invention, the method further comprises a step of storing the peptides overnight after the step of digesting proteins and before the step of labeling. In some of these embodiments, the method further comprises the step of: mixing a second unlabeled portion of peptide fragments with the labeled first portion of peptide fragments.

Some embodiments of this invention are directed to a method of quantitatively analyzing proteins, comprising the steps of: obtaining a protein sample; then dissolving the protein sample in a solution of buffer or chaotropic agent; then forming a peptide sample by digesting the protein sample by adding one or more of trypsin, chymotrypsin, Lys-C endoprotease, Glu-C endoprotease, and/or an endoprotease that cleaves at the C-terminal side of an amino acid residue on a protein; then incubating the peptide sample at least overnight; then retrieving the peptide sample; then contacting at least a first portion of the peptide sample with said one or more of trypsin, chymotrypsin, Lys-C endoprotease, Glu-C endoprotease, and/or an endoprotease that cleaves at the C-terminal side of an amino acid residue on a protein and with H₂¹⁸O to label the at least first portion of the peptide sample with ¹⁸O. In some of these embodiments, the method further comprises the steps of preparing a second unlabeled portion of said peptide samples by contacting said second portion of the peptide sample with said one or more of trypsin, chymotrypsin, Lys-C endoprotease, Glu-C endoprotease, and/or an endoprotease that cleaves at the C-terminal side of an amino acid residue on a protein and with H₂¹⁶O; and then mixing a second unlabeled portion of said peptide samples with the labeled first portion of the peptide sample; then calculating a ratio of labeled peptides to unlabeled peptides with formula (I) or (II)
ratio={I₄−(M₄/M₀))×I₀−(M₂/M₀)×[I₂−(M₂/M₀)×I₀]+[I₂−(M₂/M₀)×I₀]}/I₀ (I)
ratio={I₄−(M₄/M₀))×I₀−(M₂/M₀)×[I₂−(M₂/M₀)×I₀]+½[I₂−(M₂/M₀)×I₀]}/I₀ (II)

where I₀, I₂ and I₄ are observed peak areas for a monoisotopic peak for peptides without ¹⁸O label, a peak 2 Da higher and peak 4 Da higher, respectively, and M₀, M₂ and M₄ are the theoretical peak areas for the monoisotopic peak for a peptide with a known composition, a peak 2 Da higher and peak 4 Da higher, respectively.

### BRIEF DESCRIPTION OF THE SEVERAL VIEWS OF THE DRAWINGS

Embodiments of this invention will now be described in detail with reference to the attached Figures, in which:

FIG. 1 shows the mechanism of double ¹⁸O incorporation in peptides;

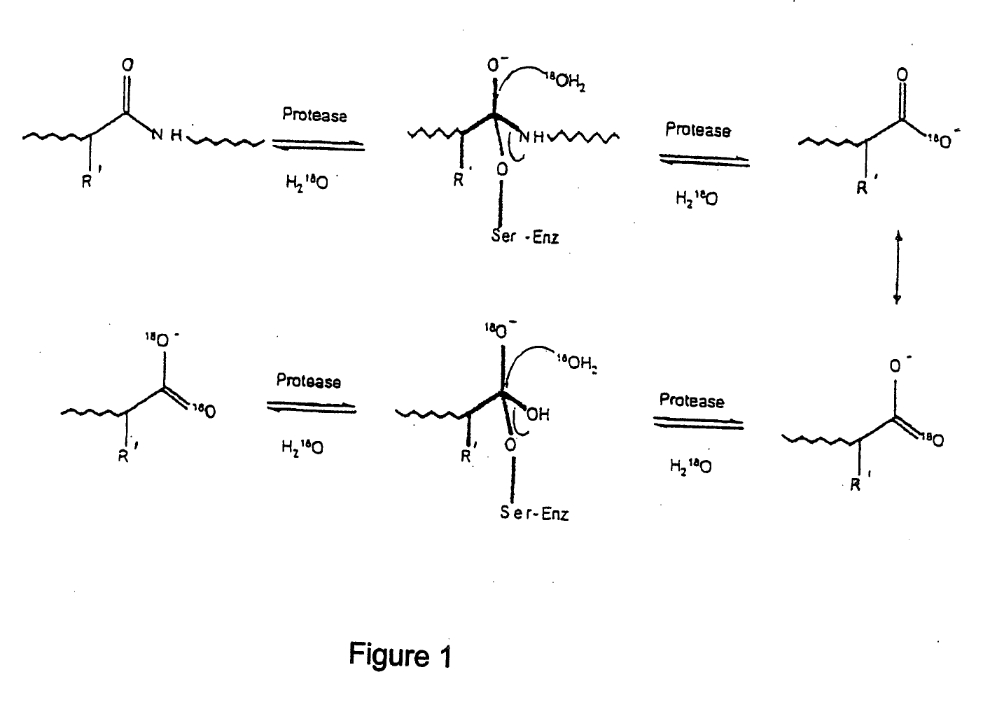

FIG. 2 shows the dissection of incorporation of two stable isotopes during proteolysis;

FIG. 3 shows MALDI-FT-ICR spectra of YGGFMK at 0% and 10% conversion of the ¹⁶O₂-peptide. Two spectra were normalized according to a total concentration of ¹⁶O₂-peptide, ¹⁶O¹⁸O-peptide, and ¹⁸O₂-peptide;

FIG. 4 shows a double reciprocal plot of 1/vᵢ vs 1/[Rₒ] for trypsin-catalyzed ¹⁶O-to-¹⁸O exchange reaction of YGGFMR;

FIG. 5 shows sequential exchange of two carboxyl oxygens by trypsin catalysis;

FIG. 6 shows the simultaneous determination of pseudo first-order decay of multiple truncated peptides (¹⁶O₂). They are YGGFMK(¹⁶O₂), YGGFLK(¹⁶O₂), and five ¹⁶O₂-peptides from apomyoglobin. R's and K's in brackets represent the peptide P₁ residues. Rate constants (k_{cat}/K_{M}'s) in the inset were calculated, using k_{cat}/K_{M} of YGGFMK as an internal standard. Data were based on a single experiment;

FIG. 7 shows sequential exchange of two carboxyl oxygens by serine protease: incorporation of the first oxygen isotope;

FIG. 8 shows MALDI FT-ICR spectra of peptides ¹⁸O₂-encoded by chymotrypsin. The last five amino acid residues to the carboxyl termini for three chymotryptic peptides are shown. Arrows point to would-be positions for the peptides with (¹⁶O₂);

FIG. 9 shows a protocol for digestion of a protein; and

FIG. 10 shows relative intensity versus mass charge for observed and theoretical isotropic distributions.

### DETAILED DESCRIPTION OF THE INVENTION

In this invention, peptides are labeled in a step separate from the proteolysis step. In one application of the invention, peptides are produced by incubation with the desired serine protease. The peptide products are dehydrated and then incubated with preferably the same type or, in some embodiments a different type of, serine protease under conditions that allow for repeated binding, and disruption of the covalent substrate/enzyme intermediate by O-18 water, sufficient to introduce two isotope labels in greater than 90%, preferably greater than 95% enrichment.

In this strategy the proteins need not be dehydrated and redissolved. Conditions for the proteolytic reaction can be optimized for proteolysis without concern for the amount of O-18 water. The incorporation of two O-18 labels may also be catalyzed by immobilized proteases, which allows for increased enzyme concentration and more rapid labeling, while controlling contamination by enzyme autolysis.

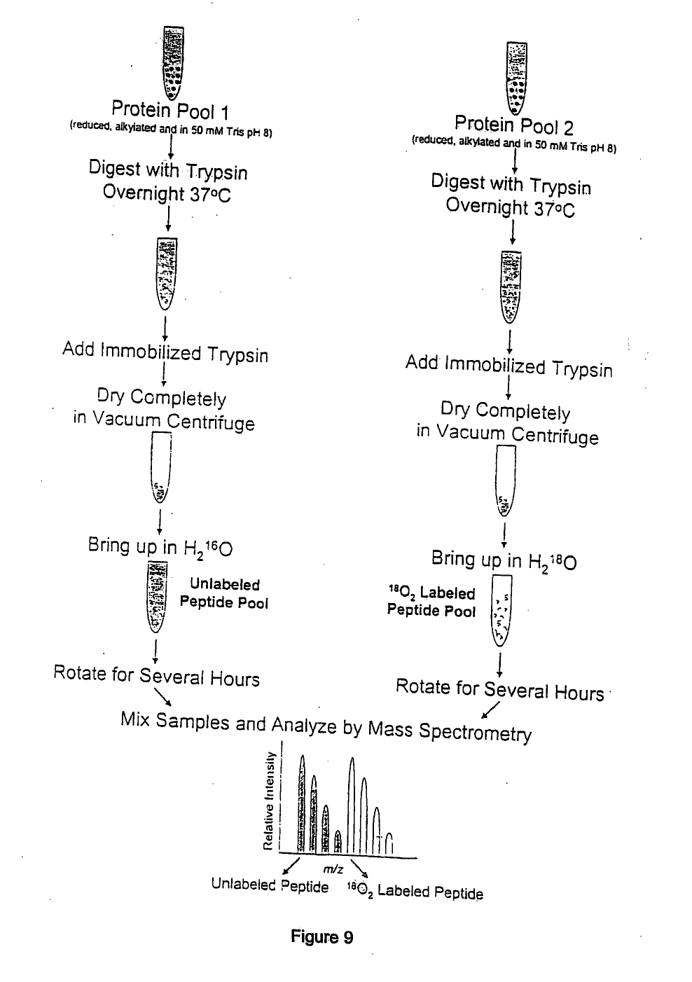

A range of times can be utilized for different peptides, reflecting the binding affinities of the enzyme for different peptides. A multiplexed kinetic study is illustrated in FIG. 9, in which incubation times for 95% exchange catalyzed by trypsin vary from about 5 minutes to about 90 min. Thus an embodiment of one standard procedure employs incubation times of about 2 hours.

As the sequencing of many species' genomes is completed, the study of the protein compliment to the genome, or the proteome, has emerged as a dynamic field of research. An approach to characterizing changed states is comparative proteomics, in which the relative amounts of proteins present in two or more samples are compared. In order to determine the relative amount of the proteins present, a proteolytic method for labeling ¹⁸O can be used.

An exemplary protocol that can be used in conjunction with embodiments of the present invention is described below:

Protocol A

Protein Sample Preparation

This protocol describes an optional step to prepare a protein sample for proteolytic labeling.

Materials

Trizma HCl (Sigma)
Trizma Base (Sigma)
Urea (Sigma)
Dithiothreitol (Sigma)
lodoacetamide (Sigma)
BioSpin 6 Column (BioRad)

Dissolve protein in 50 mM Tris (pH 8) so that the concentration is 10-200 μg/μL. For the purpose of this recipe the concentration will be 50 μg of protein in 100 μL of base. Tris of pH 8 can be made from Tris base and Tris HCl. Add 0.024 g of urea to make the urea concentration 4 M. (When trypsin is used we can be used to about 4M with a 100 M buffer concentration for Lys-C, 8M urea and for Blu-C and Chymotrypsin 2M urea can be used). Add 10 μL of 100 mM DTT and let rotate at room temperature for 1 hour. The DTT concentration is approximately 10 mM. Add 30 μL of 0.5 M lodoacetamide and let rotate at room temperature in the dark for 1 hour. The lodoacetamide concentration is approximately ten times the DTT concentration. Desalt reduced and alkylated protein sample using a BioSpin 6 Column. One spin column can desalt 75 μL, so two columns are necessary. Sample is recovered in 10 mM Tris, pH 7.4. Pool the recovered samples.

Protocol B

Initial Protein Digest

This protocol describes the steps for the digestion of the protein (FIG. 9).

Materials

Sequencing Grade Modified Trypsin (Promega)

Dissolve one vial of 20 μg of trypsin in 20 μL of 50 mM Tris (pH 8). Add 1 μL of dissolved trypsin per 50-100 μg of protein present in sample, or 0.5-1 μL in this case. Remaining trypsin can be stored and used at a later date. Check the pH to ensure that it is approximately 7-8. The pH can be adjusted by adding additional Tris. Incubate at 37° C. overnight.

Protocol C

Proteolytic H₂¹⁸O Labeling

This protocol outlines the steps for ¹⁸O labeling (FIG. 9).

Materials

Isotopically enriched H₂¹⁸O (Isotech, Inc.)
Immobilized trypsin (Applied Biosystems)

Retrieve digested protein sample from 37° C. incubator. Add 10-20 μL of immobilized trypsin to the peptide sample. Immobilized trypsin must be cleaned prior to use. Wash three times with 5X volume of water, spin on a bench top centrifuge at 8000 rpm for 1 minute and discard the supernatant each time. Dry sample completely in vacuum concentrator. Bring up in 80 μL H₂¹⁸O and 20 μL acetonitrile. The presence of the organic solvent enhances trypsin efficiency. Store H₂¹⁸O at 20° C. Rotate at room temperature for 2 to 24 hours. Mix the labeled and unlabeled peptide pools and analyze by mass spectrometry. Samples can be analyzed by nanospray, electrospray, LC-MS, or MALDI and the method is compatible with most commercial mass spectrometers so long as the resolution is sufficient to resolve 4 Da.

Protocol D

Interpretation and Protein Quantitation.

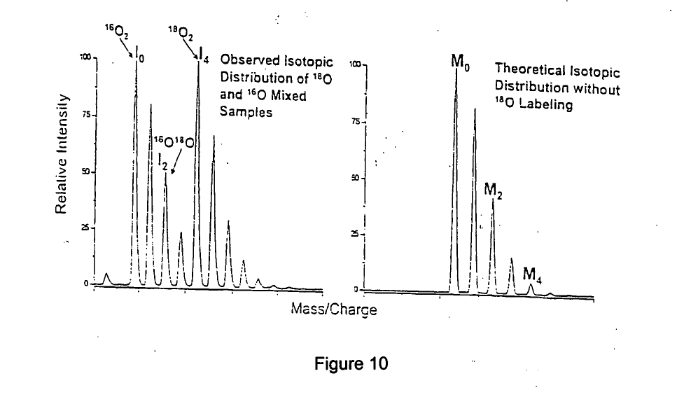

This protocol discusses the interpretation of mass spectral data in order to determine the relative protein quantitation (FIG. 10).

Materials

IsoPro 3.0

$$Ratio 1 = \frac{{I}_{4} - \frac{{M}_{4}}{{M}_{0}} {I}_{0} - \frac{{M}_{2}}{{M}_{0}} ( {I}_{2} - \frac{{M}_{2}}{{M}_{0}} {I}_{0} ) - + ( {I}_{2} \frac{{M}_{2}}{{M}_{0}} {I}_{0} )}{{I}_{0}} \qquad ( Equation 1 )$$

Ratios of (¹⁸O/¹⁶O) peptides are calculated using Equation 1, where I₀, I₂ and I₄ are the observed peak areas for the monoisotopic peak for the peptides without ¹⁸O label, the peak 2 Da higher, and the peak 4 Da higher (double ¹⁸O labeled peak), respectively. M₀, M₂ and M₄ are the theoretical peak areas for the monoisotope peak of a peptide with known composition, the peak 2 Da higher and the peak 4 Da higher, respectively. If the peptide sequence is not known an estimate of the ¹⁸O;¹⁶O ratio can be made by rationing the peak areas of I₄ and I₀. The mixing ratio can be varied from 1:1 and taken into account in the calculations. Values for M₀, M₂ and M₄ can be calculated for each peptide using IsoPro 3.0. The elemental composition and charge state are entered into the software program and the program calculates the theoretical isotopic distribution. The theoretical distribution of the unlabeled and labeled peptides is assumed to be identical.

In another experiment designed to study the time span required for tryptic peptide products of different sequences, times were determined for complete exchange of a pair of peptides identical except that one is terminated by lysine (YGGFMK) and the other by arginine (YGGFMR). The km values are 4400+−700 microM and 1300+−300 microM, respectively. The kcat values are 2800+−300 per min and 3500+−500 per min, respectively.

Embodiments of this invention are described in the paper “Dissection of Proteolytic ¹⁸O Labeling: Endoprotease-Catalyzed ¹⁶O-to-¹⁸O Exchange of Truncated Peptide Substrates” (Xudong Yao, Carlos Afonso, and Catherine Fenselau) (Journal of Proteome Research 2003, 2, 147-152), which is hereby incorporated by reference in its entirety.

Proteolytic labeling in H₂¹⁸O has been recently revived as a versatile method for proteomics research. To understand the molecular basis of the labeling process, the process has been dissected into two separate events: cleavage of the peptide amide bonds and exchange of the terminal carboxyl oxygens.

It has been demonstrated that both carboxyl oxygens can be catalytically labeled, independent of the cleavage step. Reaction kinetics of the tryptic ¹⁶O-to-¹⁸O exchange of YGGFMR, YGGFMK, and the tryptic digest of apomyoglobin were studied by matrix-assisted laser desorption/ionization Fourier transform ion cyclotron resonance mass spectrometry. A larger K_{M} for the Lys-peptide (4400±700 μM), when compared to that of the Arg-peptide (K_{M} 1300±300 μM), was mainly responsible for the slower reaction with YGGFMK (k_{cat}/K_{M} 0.64±0.14 μM⁻¹min⁻¹) compared to YGGFMR (k_{cat}/K_{M} 2.6±0.9 μM⁻¹min⁻¹).

Multiplexed kinetic studies showed that endoprotease-catalyzed oxygen exchange is a general phenomenon, allowing homogeneous ¹⁸O₂-coding of a variety of peptides. It was demonstrated for the first time that chymotrypsin ¹⁸O₂-codes peptides during proteolysis.

Proteolytic ¹⁸O labeling can be advantageously decoupled from protein digestion, and endoproteases can be used in a separate step to ¹⁸O₂-code peptides for comparative studies after proteolysis has taken place.

Tracking the changes in all of the proteins expressed by a cell or tissue in different states provides insight into life processes at the molecular level. Methods enabling the comparison of such changes proteomewide are therefore of great biological and chemical interest. The use of differential stable isotope labeling to code counterpart proteomes in comparison, coupled with mass spectrometric analysis, is one of the most promising approaches. These isotope codes can be incorporated metabolically, chemically, and enzymatically.

Universal and highly specific proteolytic labeling of proteins in H₂¹⁸O allows for various applications in proteomic studies, as well as traditional applications in mechanistic studies and sequencing of peptides by mass spectrometry. In comparative proteomics strategies, endoproteases cleave the first pool of proteins in H₂¹⁸O, and the second pool in H₂¹⁶O.⁴

The enzymes differentially label the resulting peptides at the carboxyl termini, and generate a mass difference of 4 Da. The isotope ratios of each peptide pair in the combined peptide mixtures are then measured by mass spectrometry. Signal intensities of paired peptides provide relative quantitation of their precursor proteins in the different pools; unpaired peptide signals indicate potential changes in amino acid composition, caused by mutation or co/post-translational modifications.

Homogeneity and completeness of labeling are beneficial to the successful application of this strategy. Trypsin, Lys-C, and Glu-C can incorporate two ¹⁸O atoms into the carboxyl termini of all peptides, except the original protein carboxyl termini. By contrast, some reports have indicated that lysine-terminated peptides do not incorporate two oxygen labels efficiently.^{8b-d} Other serine proteases, such as chymotrypsin, have been proposed to incorporate only one ¹⁸O during digestion of proteins in H₂¹⁸O.⁷

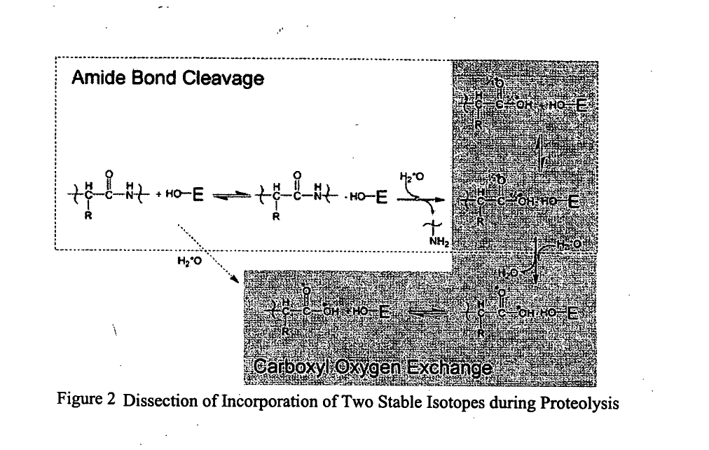

In this invention, the process has been mechanistically dissected into two separate events: cleavage of the peptide amide bond and exchange of the terminal carboxyl oxygens (FIG. 2).

Thus, if an endoprotease can catalyze both the hydrolysis and the exchange reactions, then it is able to incorporate two ¹⁸O atoms from H₂¹⁸O during the process, one at each event. The kinetics and mechanisms of the first incorporation do not differ from those of the familiar tryptic cleavage of peptide amide bonds, ignoring isotope effects caused by ¹⁸O.

Therefore, the second incorporation-the exchange reaction-is a key to understanding the molecular basis of the labeling process. Without the exchange reaction, the incorporation would end at one ¹⁸O atom.

Examples have been examined for the catalytic exchange with both trypsin and chymotrypsin.

Trypsin cleaves peptide amide bonds following arginine (Arg) or lysine (Lys) residues and is the most commonly used protease in proteomics research. The second step, the catalytic exchange, requires trypsin to recognize Arg- and Lys-terminated peptides as substrates. Compared to native peptide substrates, they are truncated and do not have any subsequent Pᵢ′ residues. (Amino acid residues from N-terminal to C-terminal of the polypeptide substrate are assigned as . . . , P₅, P₄, P₃, P₂, P₁, P₁′, P₂′, P₃′, P₃′, P₅′, . . . ,; their respective binding sub-sites in enzyme as . . . , S₅, S₄, S₃, S₂, S₁, S₁′, S₂′, S₃′, S₄′ S₅′ . . . . The enzymatic cleavage of the amide bond happens between P₁ and P₁′.)

In embodiments of this invention, YGGFMR, YGGFMK, YGGFLK, and the tryptic digest of apomyoglobin have been used to model Arg-peptides, Lys-peptides, and tryptic peptide mixtures, respectively. The reaction kinetics of the tryptic ¹⁶O-to-¹⁸O exchange of these substrates were directly studied by matrix-assisted laser desorption/ionization (MALDI) Fourier transform ion cyclotron resonance (FT-ICR) mass spectrometry (MS). The possibility was explored that both carboxylate oxygen atoms could be catalytically exchanged, independent of an amide cleavage step.

It has been demonstrated that peptide labeling can be decoupled from protein digestion for comparative studies of protein mixtures. In addition, it is demonstrated that chymotrypsin is capable of ¹⁸O₂-coding peptide carboxyl-termini.

### Experiment

Unless otherwise mentioned, all chemicals were reagent grade or better and were purchased commercially and used without further purification. The water (H₂¹⁶O) used in all experiments was purified by a Millipore (Bedford, Mass.) water filtration system. Isotopically enriched H₂¹⁸O, greater than 95%, was purchased from Isotech, Inc. (Miamisburg, Ohio). Sequencing grade modified trypsin was purchased from Promega (Madison, Wis.), and the trypsin concentration was calculated based on weight without active-site titration. Apomyoglobin (horse, muscle), α-chymotrypsin (Bovine pancreas), and peptide VEPIPY were purchased from Sigma (St. Louis, Mo.). Peptides YGGFM, YGGFMK, YGGFLK, and YGGFMR were purchased from Bachem (Philadelphia, Pa.).

Kinetics Measurement of ¹⁶O-to-¹⁸O Exchange of YGGFMK and YGGFMR Catalyzed by Trypsin. For a typical trypsin-catalyzed ¹⁶O-to-¹⁸O exchange reaction, peptide YGGFMK (2 mM) was dissolved in H₂¹⁸O (pH 8.0) containing 18 mM Tris/HCl, 45 mM CaCl₂, and the exchange reaction was initialized by addition of trypsin suspended in H₂¹⁸O to a final concentration of 36 nM (for kinetic study of peptide YGGFMR, final trypsin concentration was typically 4.5 nM). Aliquots of the reaction mixture were taken at different time intervals for the first 10% of reactant conversion. The reaction temperature was maintained at 26±1° C., and the ionic strength was controlled by NaCl (45 mM). The reaction volume was typically 20 μL. The change of the peak intensity for the peptide was monitored by MALDI-FT-ICR-MS. Substrate concentrations used in the kinetics studies ranged between 0.2 K_{M} and 5 K_{M}, and measurements were duplicated for each substrate concentration. At high concentrations (>10 mM) of YGGFMK, acetonitrile (10% v/v) was used to increase the substrate solubility in reaction buffer. A full course of ¹⁶O-to-¹⁸O exchange reaction of YGGFMR was also recorded using similar conditions.

Simultaneous Kinetic Study of ¹⁶O-to-¹⁸O Exchange of a Peptide Mixture Catalyzed by Trypsin. A mixture of tryptic apomyoglobin peptides (¹⁶O₂) (100 μM each, obtained by tryptic digestion of apomyglobin in H₂¹⁶O-buffer), YGGFMK(¹⁶O₂) (300 μM), and YGGFLK(¹⁶O₂) (300 μM) was dissolved with DMSO and then diluted with H₂¹⁸O-containg buffer (pH 7.0). The exchange reaction was initialized by the addition of trypsin solution in H₂¹⁸O. The final reaction conditions were 36 nM of trypsin, 18 mM of Tris, 45 mM of CaCl₂, 45 mM of NaCl, and 10% v/v of DMSO. Reaction was run at 26±1° C. At different time intervals, aliquots of the reaction mixture were quenched with 70:30 acetonitrile:water (0.1% TFA) with a dilution ratio of 10 times. Using MALDI-FT-ICR-MS, the changes of the peak intensities for the peptides were monitored for the whole reaction course at their corresponding m/z values.

Chymotryptic Digestion of Apomyoglobin in H₂¹⁸O. An apomyglobin (51 nmol) suspension in H₂¹⁸O (24 mM Tris/HCl, pH 8.0) was incubated with 200 μg of α-chymotrypsin (bovine pancreas, Sigma) at room temperature. Aliquots were taken at different time intervals for MALDI-FT-ICR analysis.

MALDI-FT-ICR Mass Spectrometric Analysis. Reaction mixtures in H₂¹⁸O were diluted with 70:30 acetonitrile:water (0.1% TFA) to obtain a final peptide concentration of about 10 pmol/μl. A 0.6 μL aliquot of this solution was applied on the MALDI stainless steel probe followed by 0.6 μL of a solution of 2,5-dihydroxybezoic acid (500 mM) in 70:30 acetonitrile:water (0.1% TFA). The mixture was gently stirred during the crystallization. An FT-ICR instrument equipped with a 4.7 T actively shielded magnet (lonspec Co., Irvine, Calif.) was used for this study. An external MALDI ion source was differentially pumped. A pulsed (5 ns width) Nd:YAG laser (Minilite, Contimuum, Inc., Santa Clara, Calif.) was operated at 355 nm (triple of its fundamental frequency). The laser energy was attenuated by adjusting the angle between two Glan-laser prism polarizers (Karl-Lambrecht Co., Chicago, Ill.). Ions were injected into the ICR cylindrical cell by an RF-only quadrupole ion guide. A 4 ms nitrogen pulse was used to trap the ions and remove their excess of kinetic energy. After a 4 s delay, the ions were excited by a 35 Vb-p RF sweep during 4 ms (29-180 kHz). The image signal was amplified and digitized at an acquisition rate of 2 MHz (512 k data point) resulting in the recording of a 262.1 ms time domain, which gave a typical mass resolution of 10 000. The average of single time domain transients (more than 10 times) was Fourier transformed into the corresponding frequency domain (two zero fills and Blackman apodization). In mass spectra, heights for the monoisotope peaks for peptides, the peaks with masses 2 Da higher, and the peaks with masses 4 Da higher were measured as peak intensities I₀, I₂, and I₄, respectively.

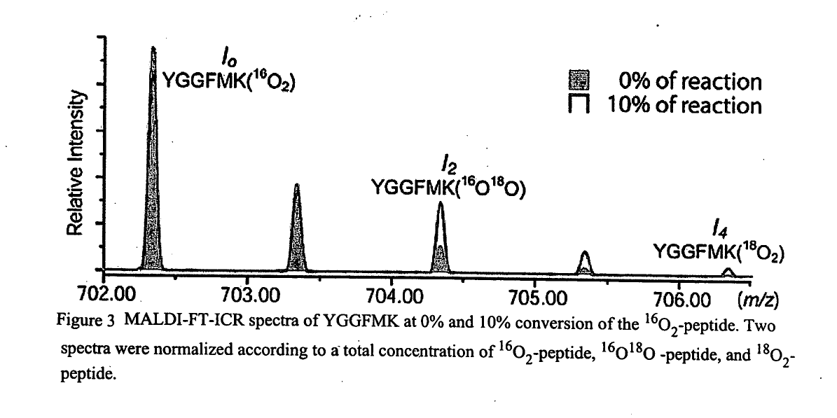

MALDI-FT-ICR Mass Spectrometric Analysis of ¹⁶O-to-¹⁸O Exchange Reactions. A relatively short detection time of 262.1 ms for recording the induced current from the coherent motion of ions in the ICR cell gave isotopic distributions close to theoretical ones. Averaging single time domain transients before the Fourier transform resulted in excellent reproducibility (better than ±3%) for the isotope distribution measurement. The concentration of reactant, [R], at any reaction time was calculated according to the following equation: [R]=[R₀]/I₀/{I₀+(I₂−M₂I₀/M₀)/2+[I₄−M₄I₀/M₀−M₂/M₀(I₂−M₂I₀/M₀)]}, where [R₀] is the initial concentration of the reactant; I₀, I₂, and I₄ are peak intensities, as assigned for YGGFMK in FIG. 3; M₀, M₂, and M₄ are the theoretical relative intensities for the monoisotope peaks for peptides, the peaks with masses 2 Da higher, and the peaks with masses 4 Da higher, which were calculated using Isopro 3.0 (lonSource.com). The residual ¹⁶O content in H₂¹⁸O (less than 5%) was not considered in formulation of the equation. Similar treatment was applied to data for YGGFMR and the apomyoglobin peptide mixture.

Kinetic Constants for ¹⁶O-to-¹⁸O Exchange of YGGFMK and YGGFMR Catalyzed by Trypsin. As discussed above, the catalytic exchange was examined using trypsin and peptides that are terminated by K or R. The exchange reactions were monitored by following the diminishing of the I₀ peak and the increase of the I₂ and I₄ peaks (FIG. 3) with time, via MALDI-FT-ICR. The contributions to higher masses at time=0 are made by natural isotope abundance. The time course for the first 10% of the reaction was limited to less than 30 min by adjusting the concentration of trypsin. Partial fragmentation of a peptide during MALDI-FT-ICR analysis did not compromise quantitation. When a fragment signal is strong, following the mass change of the fragment containing the carboxyl terminus gives the identical kinetics as following the intact peptide (data not shown).

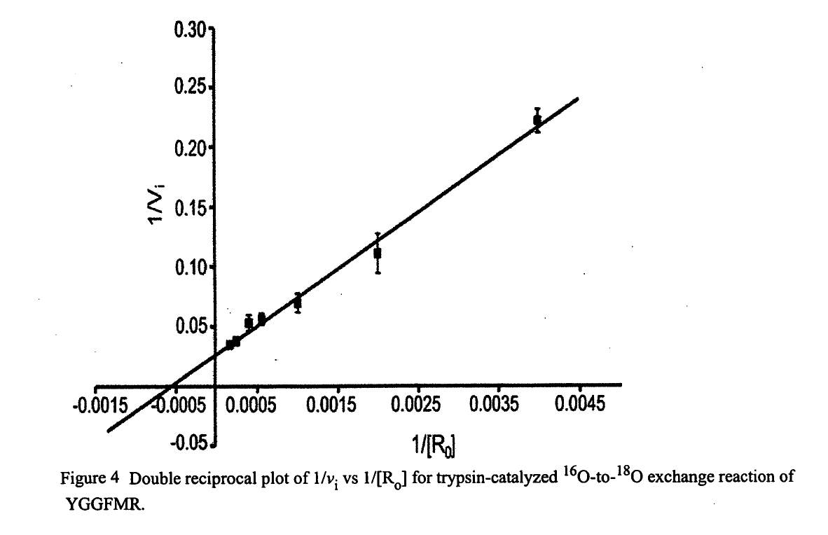

Plots of reactant concentration [R] vs reaction time for the ¹⁶O-to-¹⁸O exchange reactions were linear, giving slopes twice the initial reaction rates (2×vᵢ's). Double reciprocal plots of 1/vᵢ vs 1/[Rₒ] were linear for both peptides, and the plot for YGGFMR is shown in FIG. 4. From the plots, values for k_{cat}'s, K_{M}'s, and k_{cat}/K_{M}'s were calculated. Peptide YGGFMK has a k_{cat} of 2800±300 min⁻¹, a K_{M} of 4400±700 μM and a k_{cat}/K_{M} of 0.64±0.14 μM⁻¹min⁻¹. Peptide YGGFMR has a k_{cat} of 3500±500 min⁻¹, a K_{M} of 1300±300 μM and a k_{cat}/K_{M} of 2.6±0.9 μM⁻¹min⁻¹. High substrate concentrations (larger than 2 mM) were necessary to make the plots, especially for peptide YGGFMK. Under these concentrations, only minute amounts of coupling products (e.g., YGGFMKYGGFMK) of the peptides were observed, and quantitative correction for this side-reaction was not made. Using pseudo first-order kinetics (initial reactant concentration was smaller than one-tenth of K_{M}), k_{cat}/K_{M}'s ratios were also measured to be 0.41±0.01 μM⁻¹min⁻¹ for YGGFMK and 2.3±0.3 μM⁻¹min⁻¹ for YGGFMR, in good agreement with values from the double reciprocal plots.

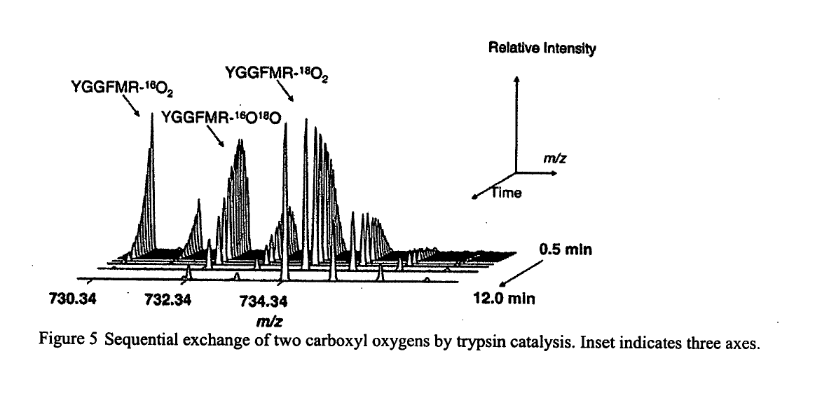

In FIG. 5, a full time course of the exchange reaction for peptide YGGFMR shows the decrease of I₀ with time, an increase up to a maximal followed by decrease for I₂ and the increase to a plateau for I₄. I₀ is qualitatively proportional to the concentration of YGGFMR(¹⁶O₂); I₂ to the concentration of YGGFMR(¹⁶O¹⁸O), the single incorporation product; I₄ to YGGFMR(¹⁸O₂), the double incorporation product.

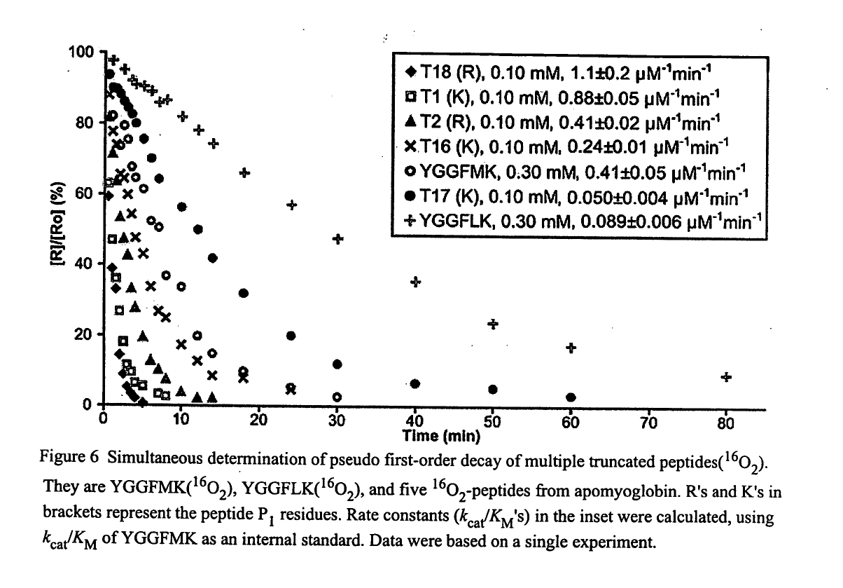

Competitive ¹⁶O-to-¹⁸O Exchange Reactions of Truncated Peptide Substrates. The tryptic digest of apomyoglobin was mixed with YGGFMK and YGGFLK, and the exchange reaction of the mixture, catalyzed by trypsin, was analyzed by MALDI-FT-ICR. In the mass range of 700 to 2000 Da, apomyoglobin peptides ALELFR (T18), HPGDFGADAQGAMTK (T17), VEADIAGHGQEVLIR (T2), GLSDGEWQQVLNVWGK (T1), and YLEFISDAIIHVLHSK (T16) could be followed to study the reaction kinetics. Other apomyoglobin peptides in the mass range overlapped with fragment ions from heavier peptides. This prevented accurate kinetic analysis. The disappearances of the ¹⁶O₂-forms of the peptides from the mixture (FIG. 6) were fitted to pseudo first-order kinetics, and the observed rate constants were obtained. From these constants, the k_{cat}/K_{M} values for other peptides (shown in the inset of FIG. 6) in the mixture were then calculated, using the k_{cat}/K_{M} value (0.41 μM⁻¹min⁻¹) for YFFFMK as an internal standard.

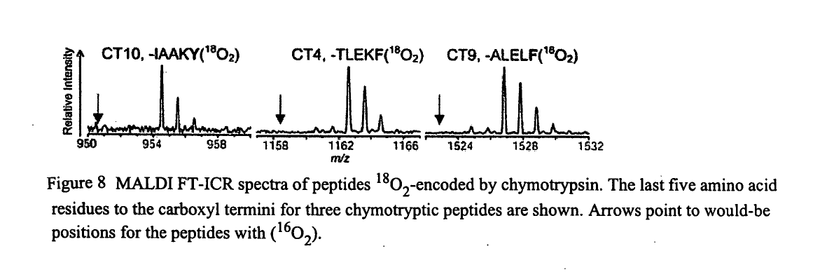

Exchange Reactions Catalyzed by Chymotrypsin. Chymotryptic proteolysis of apomyoglobin in H₂¹⁸O was analyzed by MALDI-FT-ICR. In the spectra shown in FIG. 8 some of the peptide products of this incubation, RNDIMKY (CT10), TGHPETLEKF (CT4), and GADAQGAMTKALELF (CT9), can be seen to have monoisotopic masses 4 Da higher than the theoretical ones (MH^{+:} 949.50, 1158.58 and 1522.76, respectively). This indicates incorporation of two ¹⁸O atoms into each peptide. Analysis of the fragments from these ions supported association of the labels with the carboxyl termini. Complete ¹⁸O₂-coding was obtained in 1.5 h for CT10, 3.5 h for CT4, and 20 h for CT9 (see the Experimental section).

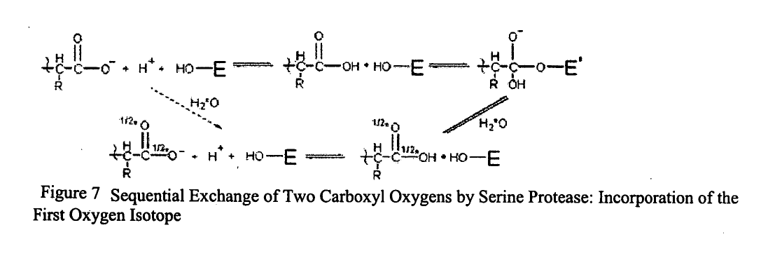

Nature of Trypsin-Catalyzed Oxygen Exchange. A minimal mechanism for the exchange reaction is shown in FIG. 7, analogous to the mechanism of serine protease catalysis for cleavage of peptide amide bonds. After formation of the acyl-enzyme adduct between the peptide and the enzyme, a molecule of water hydrolyzes the intermediate and introduces an ¹⁸O atom.

FT-ICR mass spectrometry allows direct and accurate measurement of the initial rates for the exchange reaction, in which isotopic distributions of the reactant (¹⁶O₂-peptides) and the product (¹⁶O¹⁸O-peptides and ¹⁸O₂-peptides) overlap (FIG. 3). During the first 10% of the reaction, where the initial rates were measured, changes in the intensities of I₂ and I₄ were very small. In addition, the intensity was very low for I₄ relative to that of I₀ (FIG. 3).

The difference in k_{cat} values for YGGFMK (2800±300 min⁻¹) and YGGFMR (3500±500 min⁻¹) was minimal. Weaker substrate binding for the Lys-peptide (K_{M} 4400±700 μM) compared to the Arg-peptide (K_{M} 1300±300 μM) was mainly responsible for the slower reaction with YGGFMK (k_{cat}/K_{M} 0.64±0.14 μM⁻¹min⁻¹), compared to YGGFMR (k_{cat}/K_{M} 2.6±0.9 μM⁻¹min⁻¹). Linearity of double reciprocal plots of 1/vᵢ vs 1/[R₀] for both the peptides (FIG. 4 for peptide YGGFMR) suggests that substrate/product inhibition was not significant under the experimental conditions. Equilibrium constants are compatible with K_{M}'s for chymotrypsin-catalyzed oxygen exchange reactions. Analogous to this example, we assumed that K_{M} values for the trypsin reactions gave reasonable estimations for substrate binding. Lacking all Pᵢ′-Sᵢ′ interactions, the affinity of trypsin for truncated substrates is intrinsically lower than that for larger polypeptide substrates (with both Pᵢ and Pᵢ′ sites). Hydrolysis of a native polypeptide substrate by trypsin has been reported to occur with a higher k_{cat}/K_{M} (18 μM⁻¹min⁻¹), lower K_{M} (140 μM) and similar k_{cat} (2500 min⁻¹). For very short, truncated peptide substrates, some Pᵢ-Sᵢ interactions are missing in addition to nonexisting Pᵢ′-Sᵢ′ interactions; and the exchange reaction was even slower (data not shown). Mass spectra of such short peptides do not typically provide unambiguous identification of their precursor proteins.

The fact that trypsin catalyzes the exchange reaction of both carboxyl oxygens of YGGFMK with oxygen atoms from water proves that the protease is able to ¹⁸O₂-code peptides that end in lysine. If the isotope effects from ¹⁸O are omitted, then the rates of both incorporations are identical. Previous observations that only a single ¹⁸O could be incorporated into Lys-terminated peptides during proteolysis of proteins in H₂¹⁸O^{8b-d} appear to have resulted from nonideal reaction conditions, e.g., insufficient enzyme concentration or insufficient reaction time. In the case where the labeling process starts with the digestion of proteins, diffusion of proteins or incomplete cleavage fragments to the enzyme active site could present a rate-limiting step.

For the process of ¹⁸O₂-coding of Arg- and Lys-terminated peptides through two exchange reactions, a sequential mechanism (FIG. 7) is supported by the kinetics shown in FIG. 5. Trypsin-catalyzed exchange reactions are substrate-specific; only peptides with Arg or Lys in the P₁ position can undergo exchanges catalyzed by trypsin. As a control experiment, a mixture of VEPIPY (710 μM) and YGGFM (500 μM) was incubated with 200 mM Tris and 2 μg trypsin in H₂¹⁸O (pH 8.5) at room-temperature overnight. No detectable exchange of oxygens, even the first oxygen exchange, was observed. Thus, there was not significant catalysis from either the enzyme or the buffer.

Kinetic Study of Simultaneous Exchange Reactions of Peptide Mixtures. The time required for complete ¹⁸O exchange of the mixture multiplexed in this experiment was less than 2.5 h, and k_{cat}/K_{M} ratios varied from 0.089±0.006 μM⁻¹min⁻¹ for YGGFLK to 1.1±0.2 μ⁻¹min⁻¹ for ALELFR (T18 of apomyoglobin), which further demonstrates the general applicability of the exchange reaction. The range of k_{cat}/K_{M} ratios for a peptide mixture can vary by several orders of magnitude, as estimated using data from a study on trypsin subsites. Furthermore, this study provides an example of the simultaneous study of multiple reactions, using the capabilities of MALDI-FT-ICR: large dynamic range, ultrahigh resolution, and the fact that MALDI predominantly generates singly charged ions.

There are often peptides (e.g., hydrophobic peptides) in a mixture that are difficult to dissolve directly in aqueous buffers. When a peptide was not fully dissolved in solution, two populations of the peptide (¹⁶O₂-peptide and ¹⁸O₂-peptide, 4 Da difference in mass) were observed in mass spectrometric analysis. This suggests that after reaching steady-state (all dissolved molecules of a peptide having exchanged their carboxyl terminal oxygen atoms), solubilization of the peptide is the rate-limiting step in the labeling reaction. Before reconstitution of peptide mixtures with H₂¹⁸O buffers for the exchange reaction, dissolution of the entire mixture with a strong solvent such as DMSO is beneficial to successful homogeneous labeling.

The use of fluorescent peptide libraries has been recently reported to provide high-throughput profiling of protease specificities for peptides with different Sᵢ and Sᵢ′ sites. An alternative strategy can be provided by ¹⁶O-to-¹⁸O exchange reactions, which eliminates the need for conjugation of fluorescent labels. If sequences of Arg-terminated peptides are systematically changed and synthesized, then trypsin-catalyzed ¹⁶O-to-¹⁸O exchange reactions of such peptide libraries could be used to profile trypsin specificities for Sᵢ sites. Analogous experiments can be designed for other proteases. The capability of MALDI-FT-ICR to monitor multiple reactions simultaneously increases the throughput of this method.

¹⁸O₂-Coding of Peptides by Chymotrypsin. It is demonstrated here that chymotrypsin can ¹⁸O₂-code peptides during proteolysis. The different times for complete ¹⁸O₂-coding appear to reflect recognition preferences of the enzyme active site for the carboxyl terminal sequences of the peptides. Chymotrypsin and other serine endoproteases are known to catalyze oxygen exchange of carboxylic acids. However, chymotrypsin was believed to label peptide termini with only one ¹⁸O during proteolysis in H₂¹⁸O. The mechanistic dissection of the proteolytic labeling process reveals this observation to be experimentally dependent, not reflecting the intrinsic catalytic capability of chymotrypsin. Compared to trypsin, chymotrypsin catalysis of oxygen exchange reactions was less efficient, but with sufficient enzyme concentration, as can be achieved by immobilized forms of the enzyme, the exchange could be completed on a practical time scale. Recognizing large hydrophobic residues as P₁ sites, this enzyme could be useful in comparative proteomic studies of hydrophobic protein mixtures.

It is demonstrated above that various endoproteases catalyze the oxygen exchange reaction of appropriate truncated peptides, independent of the cleavage of peptide amide bonds in the precursor proteins. (On the basis of mechanistic similarity, all of the serine proteases, if not other classes of proteases, that cleave C-side of P₁ residues should have the same capability. Trypsin, chymotrypsin, Lys-C, and Glu-C are among these proteases.) Therefore, proteolytic ¹⁸O labeling can be decoupled from protein digestion and to use endoproteases to ¹⁸O₂-code peptides after proteolysis.

In this strategy, the two protein mixtures being compared are digested separately in H₂¹⁶O under optimal conditions and dried. Each resulting peptide mixture is then reconstituted with H₂¹⁶O or H₂¹⁸O for enzyme-catalyzed differential labeling with stable isotopes of oxygen. After combination of the two pools of labeled peptides, concurrent separation and mass spectrometric characterization will provide accurate comparisons of changes in expression and modification of proteins in the counterpart mixtures. It is worthwhile to discuss that although MALDI-FT-ICR was used in quantitative study of oxygen exchange kinetics, other mass spectrometers can serve well for peptide/protein quantification enabled by ¹⁸O/¹⁶O-labeling. In fact, the resolution for FT-ICR was set to be ca. 10 000, which is in the resolution range for mass spectrometers with configurations such as quadrupole-time-of-flight, reflectron-time-of-flight or triple quadrupole. When doubly or triply charged ions of differentially labeled peptides have small mass difference as 3 or 4 Da, use of low-resolution mass spectrometers such as ion traps can be difficult; a new mass spectrometric method has been proposed to address this issue.

The decoupling has the advantages that proteins can be kept in solution prior to digestion, that proteolytic peptide products can be labeled with a limited volume of H₂¹⁸O, and that digestion and labeling conditions can be optimized separately. All of these features are of real interest in investigations of complex protein mixtures, and they allow more effective and versatile applications of enzymatic labeling. This decoupling strategy can be used, for example, to study changes of proteins in human cancer cells upon acquisition of drug-resistance.

It has been shown that chymotrypsin can catalyze full exchange of two O-18 atoms into peptides recovered from a separate chymotrypsin proteolysis step, as indicated in FIG. 10. The reaction time is slower for catalysis by chymotryptic than for tryptic catalysis, however, this exchange based on binding and on the mechanism of the enzyme.

A method in accordance with embodiments of this invention has been utilized for investigating doxorubicin resistance in MCF-7 Breast Cancer cells (see the paper “Investigation of Doxorubicin Resistance in MCF-7 Breast Cancer Cells Using Shot-Gun Comparative Proteomics with Proteolytic 18O Labeling, Brown and Fensalau, Journal of Proteome Research, Received Oct. 6, 2003). The subject matter of that paper is incorporated herein in its entirety.) In that paper, the decoupling of the digestion and labeling steps eliminated the need to dry samples at the protein level. To further optimize the ¹⁸O labeling strategy, the use of immobilized enzymes was implemented. This strategy was optimized for the application to complex protein mixtures. The use of an immobilized enzyme allowed for a high enzyme concentration with minimal autolysis products. This extremely high enzyme concentration allowed for a shorter reaction time and more complete incorporation.

1. A method of labeling peptides, comprising the steps of:
a) obtaining peptides formed from proteins digested by proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes; and thereafter
b) incorporating isotopic atoms into said peptides in the catalytic presence of proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes.

2. The method of claim 1 wherein the proteolytic enzymes or mixture of enzymes in step a) is the same as the proteolytic enzymes or mixture of proteolytic enzymes in step b).

3. The method of claim 2 wherein the proteolytic enzymes in step a) and in step b) are trypsin.

4. The method of claim 2 wherein the proteolytic enzymes in step a) and in step b) are a mixture of Glu-C and trypsin.

5. The method of claim 1 wherein the proteolytic enzymes or mixture of enzymes in step a) are different from the proteolytic enzymes or mixture of proteolytic enzymes in step b).

6. The method of claim 5 wherein the proteolytic enzymes in step a) are Lys-C and in step b) are trypsin.

7. The method of claim 1 wherein said the proteolytic enzymes in step a) and in step b) can be obtained from any species or from thermophilic microorganisms.

8. The method of claim 1 wherein one or two isotopic atoms are incorporated into a peptide and where said isotopic atoms are ¹⁸O atoms.

9. The method of claim 8 wherein step of incorporating isotopic atoms into said peptides is conducted in H₂¹⁸O.

10. The method of claim 1, further comprising mixing labeled peptides obtained from the step of incorporating of isotopic atoms with peptides that have not been labeled to obtain a mix of labeled and unlabeled peptides.

11. The method of claim 10, further comprising analyzing the mix of labeled and unlabeled peptides by mass spectrometry.

12. The method of claim 11, further comprising calculating a ratio of labeled peptides to unlabeled peptides with formula (I) or (II)
ratio−{I₄−(M₄/M₀))×I₀×(M₂/M₀)×[I₂−(M₂/M₀)×I₀]+[I₂−(M₂/M₀)×I₀]}/I₀ (I)
ratio={I₄−(M₄/M₀))×I₀−(M₂/M₀)×[I₂−(M₂/M₀)×I₀]+½[I₂−(M₂/M₀)×I₀]}I₀ (II)
where I₀, I₂ and I₄ are observed peak areas for a monoisotopic peak for peptides without ¹⁸O label, a peak 2 Da higher and peak 4 Da higher, and M₀, M₂ and M₄ are the theoretical peak areas for the monoisotopic peak for a peptide with a known composition, a a peak 2 Da higher and peak 4 Da higher, respectively.

13. The method of claim 11, wherein said mass spectrometry is nanospray, electrospray, LC-MS, LC-MS-MS or matrix-assisted IR or UV laser desorption ionization, high vacuum, atmospheric or low pressure, on a quadropole, quadropole ion trap, time-of-flight, ion cyclotron resonance, magnetic sector ion analyzer or a combination thereof.

14. The method of claim 1, wherein said step of incorporating isotopic atoms results in peptides in the catalytic presence of proteolytic enzymes being labeled greater than 90% with one or two ¹⁸O atoms.

15. The method of claim 1, further comprising a step of storing the peptides overnight after step a) and before step b).

16. A method of labeling peptides, comprising the steps of:
digesting proteins with proteolytic enzymes in an H₂¹⁶O environment to obtain peptide fragments of said proteins, where said proteolyic enzymes are selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes; and then
labeling at least a first portion of said peptide fragments by incorporating ¹⁸O atoms into said at least first portion of said peptide fragments in an H₂¹⁸O environment and in the catalytic presence of proteolyic enzymes selected from trypsin, chymotrypsin, Lys-C endoprotease, Glu-C endoprotease, an endoprotease that cleaves at the C-terminal side of an amino acid residue on a protein, or a mixture of two or more of these proteolytic enzymes.

17. The method of claim 16, further comprising a step of storing the peptides overnight after the step of digesting proteins and before the step of labeling.

18. The method of labeling peptides of claim 17, further comprising the step of:
mixing a second unlabeled portion of peptide fragments with the labeled first portion of peptide fragments.

19. A method of quantitatively analyzing proteins, comprising the steps of:
obtaining a protein sample;
then dissolving the protein sample in a solution of buffer or chaotropic agent;
then forming a peptide sample by digesting the protein sample by adding one or more of trypsin, chymotrypsin, Lys-C endoprotease, Glu-C endoprotease, and/or an endoprotease that cleaves at the C-terminal side of an amino acid residue on a protein;
then incubating the peptide sample at least overnight;
then retrieving the peptide sample;
then contacting at least a first portion of the peptide sample with said one or more of trypsin, chymotrypsin, Lys-C endoprotease, Glu-C endoprotease, and/or an endoprotease that cleaves at the C-terminal side of an amino acid residue on a protein and with H₂¹⁸O to label the at least first portion of the peptide sample with ¹⁸O.

20. The method of labeling peptides of claim 19, further comprising the steps of:
preparing a second unlabeled portion of said peptide samples by contacting said second portion of the peptide sample with said one or more of trypsin, chymotrypsin, Lys-C endoprotease, Glu-C endoprotease, and/or an endoprotease that cleaves at the C-terminal side of an amino acid residue on a protein and with H₂¹⁶O; and then
mixing a second unlabeled portion of said peptide samples with the labeled first portion of the peptide sample;
then calculating a ratio of labeled peptides to unlabeled peptides with formula (I) or (II)
ratio={I₄−(M₀/M))×I₀−(M₂/M₀)×[I₂−(M₂/M₀)×I₀]+[I₂−(M₂/M₀)×I₀]}/I₀ (I)
ratio={I₄−(M₀/M))×I₀−(M₂/M₀)×[I₂−(M₂/M₀)×I₀]+½[I₂−(M₂/M₀)×I₀]}/I₀ (II)
where I₀, I₂ and I₄ are observed peak areas for a monoisotopic peak for peptides without ¹⁸O label, a peak 2 Da higher and peak 4 Da higher, respectively, and M₀, M₂ and M₄ are the theoretical peak areas for the monoisotopic peak for a peptide with a known composition, a peak 2 Da higher and peak 4 Da higher, respectively.
